(19) United States
(12) Patent Application Publication
(10) Pub. No.: US 2006/0099269 A1
(43) Pub. Date: May 11, 2006

(54) Pulmonary delivery of inhibitors of phosphodiesterase type 5

(75) Inventors: Wayman Wendell Cheatham, Columbia, MD (US); Andrea Leone-Bay, Ridgefield, CT (US); Marshall Grant, Newtown, CT (US); Per B. Fog, Bedford Hills, NY (US); David C. Diamond, West Hills, CA (US)

Correspondence Address: PRESTON GATES & ELLIS LLP, 1900 MAIN STREET, SUITE 600, IRVINE, CA, 92614-7319, US

(73) Assignee: MannKind Corporation, Valencia, CA (US)

(21) Appl. No.: 11/210,709

(22) Filed: Aug. 23, 2005

Related U.S. Application Data
(60) Provisional application No. 60/603,764, filed on Aug. 23, 2004.

(51) Int. Cl. A61K 31/519; A61K 9/14

(52) U.S. Cl. 424/489; 514/252.16; 514/262.1

(57) ABSTRACT

Provided herein are compositions of 1) diketopiperazine salts of PDE5 inhibitors, and 2) DKP microparticles having a PDE5 inhibitors thereon, as well as methods for the pulmonary delivery of these compositions for the treatment of pulmonary hypertension and sexual dysfunction(s).

### RELATED APPLICATION

The present application claims priority under 35 U.S.C. §119(e) to U.S. Provisional Patent Application No. 60/603,764 filed Aug. 23, 2004.

### FIELD OF THE INVENTION

This invention is generally in the field of treatment of pulmonary hypertension and sexual dysfunction, including erectile dysfunction and female sexual dysfunction. In particular the present invention relates to diketopiperazine salts of phosphodiesterase type 5 inhibitors. Also, the present invention relates to pulmonary administration of phosphodiesterase type 5 inhibitors, particularly substituted pyrimidinones, such as the pyrazolopyrimidinones, sildenafil and vardenafil, utilizing microparticle compositions comprising substituted diketopiperazine or polymers.

### BACKGROUND OF THE INVENTION

Sildenafil, a pyrazolopyrimidinone phosphodiesterase type 5 inhibitor (PDE5), is a widely prescribed drug with FDA approval for the treatment of erectile dysfunction (U.S. Pat. No. 6,469,012 entitled “Pyrazolopyrimidines for the treatment of impotence”). It has also been applied to female sexual dysfunction of a variety of etiologies (see, for example: Dasgupta et al., J. Urol. 171:1189-93, 2004; Laan et al., J. Womens Health Gend. Based Med. 11:357-365, 2002; Berman et al., J. Sex Marital Ther. 27:411-420, 2001; Vemulapalli and Kurowski, Life Sci. 67:23-29, 2000; Sher and Fisch, Hum. Reprod. 15:806-809, 2000; Numberg et al, Psychiatr. Serv. 50:1076-1078, 1999; Shen et al., J. Reprod. Med. 44:535-542,1999).

Sildenafil and other PDE5 inhibitors have also shown usefulness in the treatment of pulmonary hypertension (see, for example: Leuchte et al., Chest. 125:580-6, 2004; Bonnell et al., Ann. Thorac. Surg. 77:238-42, 2004; Travadi and Patole, Pediatr. Pulmonol. 36:529-35, 2003; Michelakis et al., Circulation 108:2066-9, 2003; Bhatia et al., Mayo Clin. Proc. 78:1207-13, 2003). More recently, sildenafil has received FDA approval for the treatment of pulmonary arterial hypertension (PAH). Other drugs with related chemical structures, mechanisms of action, and clinical indications include vardenafil and tadalafil.

One point of user dissatisfaction with such drugs has been the length and variability of the time needed for the drug to take effect. As marketed by their respective manufacturers, these drugs are available as orally administered tablets. Thus, the drug enters the blood stream through the digestive tract. This can require one to several hours depending in part on food consumption. Also, these orally administered tablets may be exposed to drug-drug interactions, food-drug interactions and/or they may be poorly absorbed via the gastrointestinal tract. In one attempt to overcome these problems, pharmacists have compounded lozenges and chewing gums from crushed tablets to facilitate drug absorption through the sublingual and buccal routes, but even by these routes the drugs can require at least 15-20 minutes to take effect.

In the early 1970s, it was found that certain medicines could be administered in dry powder form directly to the lungs by inhalation through the mouth or inspiration through the nose. This process allows the medicine to bypass the digestive system, and may, in certain cases, allow smaller dosages to be used to achieve the same results as orally ingested or injected medicines. In some cases, this process provides a delivery technique that reduces the side effects associated with these medicines and reduces interactions with other prescribed medicines, as well as providing a more rapid drug medication absorption and/or uptake.

Therefore a need exists for a rapidly acting pulmonary delivery system for the treatment of pulmonary hypertension and sexual dysfunction.

### SUMMARY OF THE INVENTION

The present invention provides compositions and methods for the pulmonary delivery of phosphodiesterase type 5 (PDE5) inhibitors to treat pulmonary hypertension and sexual dysfunction. Compositions according to the present invention include diketopiperazine (DKP) salts of PDE5 inhibitors and DKP microparticles associated with PDE5 inhibitors. Embodiments of the present invention provide for treating forms of sexual dysfunction including erectile dysfunction and female sexual dysfunction.

The present invention also provides compositions including compositions of diketopiperazine salts of PDE5 inhibitors including, but not limited to, substituted pyrimidinones and pyrazolopyrimidinones such as sildenafil, vardenafil, tadafinil and analogues thereof.

In an embodiment of the present invention, the diketopiperazine has the general structure: 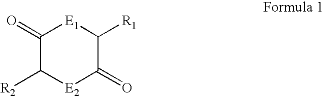
wherein ring atoms E₁ and E₂ are either O or N and at least one of R₁ and R₂ contain a carboxyl group. In another embodiment of the present invention both R₁ and R₂ contain a carboxyl group.

An embodiment of the present invention provides a diketopiperazine salt wherein the diketopiperazine is selected from the group consisting of 2,5-diketo-3,6-di(4-fumarylaminobutyl)piperazine, 2,5-diketo-3,6-di(4-sucinylaminobutyl)piperazine, 2,5-diketo-3,6-di(4-glutarylaminobutyl)piperazine, and 2,5-diketo-3,6-di(4-maleylaminobutyl) piperazine.

An embodiment of the present invention includes a diketopiperatizine salt of a PDE5 inhibitor where the ratio of the PDE5 inhibitor to the diketopiperazine salt is about 1:1 or about 2:1.

In another embodiment of the present invention, the diketopiperazine salt is formulated as a dry microparticle.

Another embodiment of the present invention includes a microparticle composition for delivery of a PDE5 inhibitor comprising diketopiperazine microparticles, wherein the microparticles are insoluble at a first defined pH and soluble at a second defined pH, and a PDE5 inhibitor or a pharmaceutically acceptable salt thereof. The PDE5 inhibitors of the present invention may be selected from the group consisting of sildenafil citrate, vardenafil hydrochloride, and tadalafil.

In an embodiment of the present invention the microparticle composition is formed by precipitation, either by freezing or chilling, of a PDE5 inhibitor or a pharmaceutically acceptable salt thereof onto diketopiperazine microparticles.

In an embodiment of the present invention, the microparticle composition is formed by spray drying diketopiperazine microparticles suspended in a solution of a PDE5 inhibitor or a pharmaceutically acceptable salt thereof.

In another embodiment of the present invention, the pharmaceutically acceptable salt is a diketopiperazine salt.

In an embodiment of the present invention, the microparticle composition of is formed by precipitation of a solution comprising a diketopiperazine and a PDE5 inhibitor or a pharmaceutically acceptable salt thereof.

A further embodiment of the present invention provides a microparticle composition for delivery of a PDE5 inhibitor to the pulmonary system comprising diketopiperazine microparticles which have a diameter between about 0.5 microns and about 10 microns and which release incorporated PDE5 inhibitor or a pharmaceutically acceptable salt thereof at a pH of about 6.0 or greater.

In another embodiment of the present invention, the microparticle composition is formulated for oral administration.

Another embodiment of the present invention provides a method of treating sexual dysfunction comprising delivering to the pulmonary system of a patient in need of treatment for sexual dysfunction, diketopiperazine microparticles comprising a PDE5 inhibitor or a pharmaceutically acceptable salt thereof. The sexual dysfunction is erectile dysfunction or female sexual dysfunction. The female sexual dysfunction is selected from the group consisting of antidepressant-induced sexual dysfunction, sexual dysfunction secondary to multiple sclerosis, anorgasmia, low arousal, delayed orgasm, decreased vaginal engorgement, dyspareunia or infertility-induced sexual dysfunction.

An embodiment of the present invention provides a method of treating pulmonary hypertension comprising delivering to the pulmonary system of a patient in need of treatment for pulmonary hypertension, diketopiperazine microparticles comprising a PDE5 inhibitor or a pharmaceutically acceptable salt thereof. The pulmonary hypertension is selected from the group consisting of primary pulmonary hypertension (PPH), acute pulmonary hypertension, pulmonary arterial hypertension (PAH), pregnancy-associated hypertension such as preeclampsia, and persistent pulmonary hypertension of the newborn (PPHN).

A method of oral delivery of a rapidly absorbed diketopiperazine formulation is also provided.

### BRIEF DESCRIPTION OF THE DRAWINGS

FIG. 1 depicts an isometric view of an exemplary inhaler suitable for delivering the compositions of the present invention to the pulmonary system.

FIG. 2 depicts the chemical structure of sildenafil citrate.

FIG. 3 depicts the chemical structure of vardenafil hydrochloride.

FIG. 4 depicts the chemical structure of the sildenafil analog UK 343-664.

FIG. 5 depicts the chemical structure of the sildenafil analog UK 347-334.

FIG. 6 depicts the chemical structure of tadalafil.

### DETAILED DESCRIPTION OF THE INVENTION

The present invention includes compositions of 1) diketopiperazine (DKP) salts of phosphodiesterase type 5 (PDE5) inhibitors, and 2) DKP microparticles having a PDE5 inhibitors associated therewith, as well as methods for the pulmonary delivery of these compositions for the treatment of pulmonary hypertension and sexual dysfunction(s).

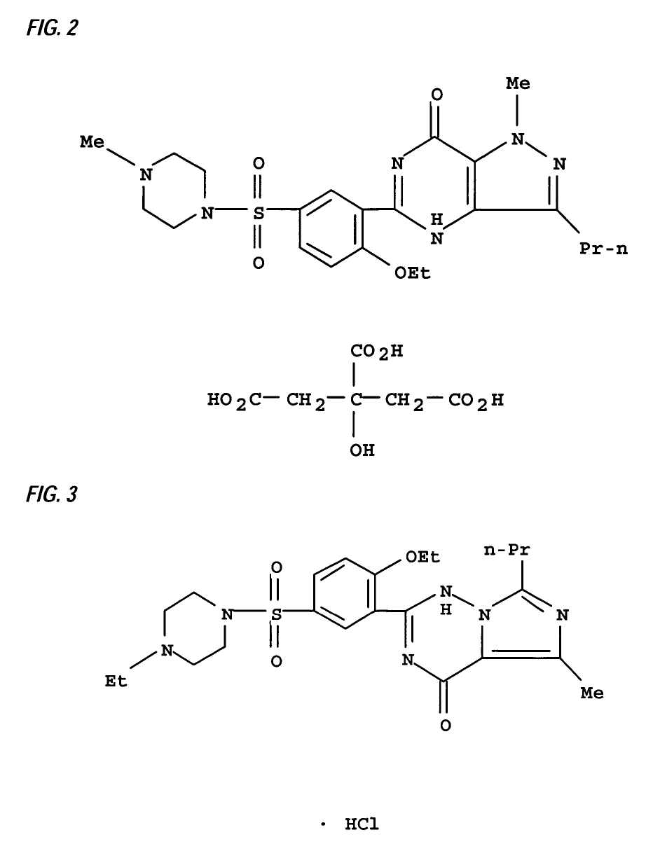

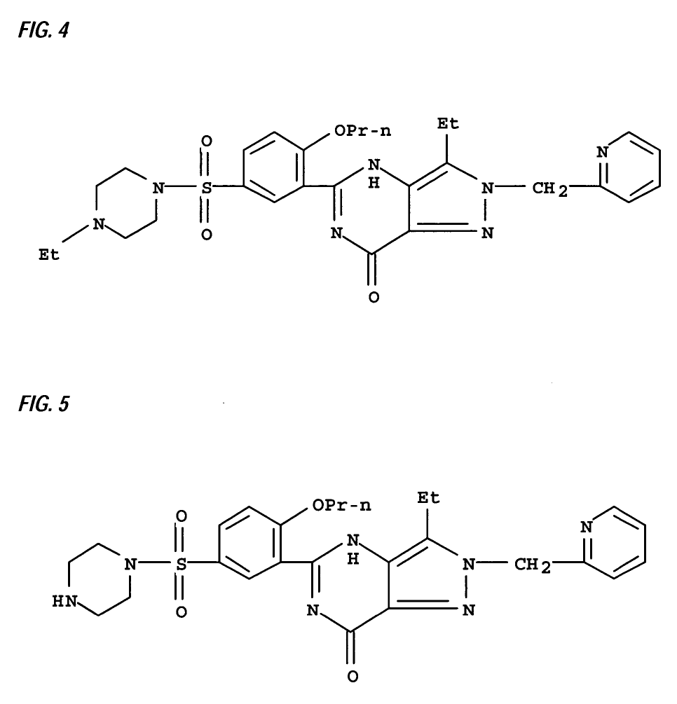

Pyrazolopyrimidinones such as sildenafil, vardenafil, UK 343-664 and UK 347-334 (see FIGS. 2, 3, 4 and 5, respectively and Table 1) are inhibitors of the enzyme cyclic guanosine monophosphate (cGMP)-specific phosphodiesterase type 5 (PDE5). Cyclic GMP is involved in the physiologic regulation of smooth muscle relaxation. Nitric oxide (NO) activates the enzyme guanylate cyclase, which forms cGMP leading in turn to smooth muscle relaxation, vasodilation and increased blood flow. PDE5 converts cGMP to GMP thereby counteracting the vasodilation brought about by cGMP. Inhibition of PDE5 increases vasodilation facilitating penile erection in males and engorgement of the endometrial and vaginal tissues in females. Similarly, vasodilation can ameliorate hypertension.

TABLE 1

| PDE5 inhibitor | Formula | CA Index name | Other names |
|---|---|---|---|
| sildenafil | C₂₂H₃₀N₆O₄S.C₆H₈O₇ | Piperazine, 1-[[3-(4,7- | 1-[[3-(6,7-Dihydro-1-methyl- |
| | | dihydro-1-methyl-7-oxo-3- | 7-oxo-3-propyl-1H- |
| | | propyl-1H-pyrazolo[4,3- | pyrazolo[4,3-d]pyrimidin-5- |
| | | d]pyrimidin-5-yl)-4- | yl)-4-ethoxyphenyl]sulfonyl]- |
| | | ethoxyphenyl]sulfonyl]-4- | 4-methylpiperazine, 2- |
| | | methyl-, 2-hydroxy-1,2,3- | hydroxy-1,2,3- |
| | | propanetricarboxylate (1:1) | propanetricarboxylate (1:1); |
| | | (9Cl) | Sildenafil citrate; UK 92480; |
| | | | UK 92480-10; Viagra |
| vardenafil | C₂₃H₃₂N₆O₄S.HCl | Piperazine, 1-[[3-(1,4- | Levitra; Vardenafil |
| | | dihydro-5-methyl-4-oxo-7- | hydrochloride |
| | | propylimidazo[5,1- | |
| | | f][1,2,4]triazin-2-yl)-4- | |
| | | ethoxyphenyl]sulfonyl]-4- | |
| | | ethyl-, monohydrochloride | |
| | | (9Cl) | |
| UK 343-664 | C₂₈H₃₅N₇O₄S | Piperazine, 1-ethyl-4-[[3-[3- | 3-Ethyl-5-[5-(4- |
| | | ethyl-4,7-dihydro-7-oxo-2-(2- | ethylpiperazin-1-ylsulfonyl)- |
| | | pyridinylmethyl)-2H- | 2-propoxyphenyl]-2-(pyridin- |
| | | pyrazolo[4,3-d]pyrimidin-5- | 2-yl)methyl-2,6-dihydro-7H- |
| | | yl]-4-propoxyphenyl] | pyrazolo[4,3-d]pyrimidin-7- |
| | | sulfonyl]-(9Cl) | one |
| UK 347-334 | C₂₆H₃₁N₇O₄S | Piperazine, 1-[[3-[3-ethyl- | n/a |
| | | 4,7-dihydro-7-oxo-2-(2- | |
| | | pyridinylmethyl)-2H- | |
| | | pyrazolo[4,3-d]pyrimidin-5- | |
| | | yl]-4-propoxyphenyl] | |
| | | sulfonyl]-(9Cl) | |
| tadalafil | C₂₂H₁₉N₃O₄ | Pyrazino[1′,2′:1,6]pyrido[3,4- | Pyrazino[1′,2′:1,6]pyrido[3,4- |
| | | b]indole-1,4-dione, 6-(1,3- | b]indole-1,4-dione, 6-(1,3- |
| | | benzodioxol-5-yl)- | benzodioxol-5-yl)- |
| | | 2,3,6,7,12,12a-hexahydro-2- | 2,3,6,7,12,12a-hexahydro-2- |
| | | methyl-, (6R,12aR)-(9Cl) | methyl-, (6R-trans)-; |
| | | | (6R,12aR)-2,3,6,7,12,12a- |
| | | | hexahydro-2-methyl-6-(3,4- |
| | | | methylenedioxyphenyl)pyrazino |
| | | | [1′,2′:1,6]pyrido[3,4- |
| | | | b]indole-1,4-dione; Cialis; GF |
| | | | 196960; IC 351; ICOS 351 |

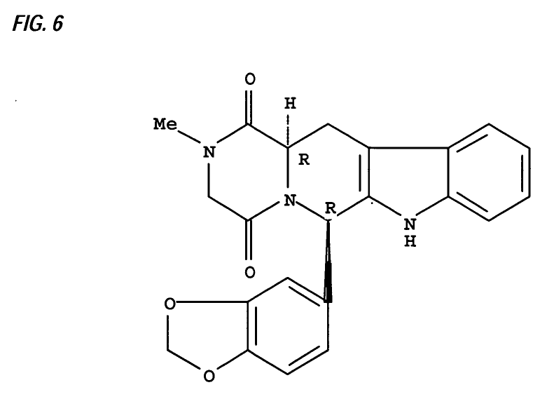

Other substituted pyrimidinone PDE5 inhibitors, such as tadalafil (FIG. 6 and Table 1), can be effective over extended periods of time, attenuating the imperative for rapid onset of effectiveness in treating sexual dysfunction. Nonetheless, rapid onset of effectiveness can still offer a measure of flexibility and convenience to the user. Such rapid onset can also be important for application to the treatment of pulmonary hypertension, particularly for acute forms.

These PDE5 inhibitors have typically been administered orally. The oral route of administration is associated with slower than optimally desired absorption resulting in delayed effectiveness. Administration of PDE5 inhibitors through the lungs facilitates improved and rapid absorption, by the large surface area afforded by the lungs. In one embodiment of the present invention, compositions are provided of DKP salts of PDE5 inhibitors. In another embodiment of the present invention, DKP microparticles are provided having PDE5 inhibitors associated therewith.

As used herein, “diketopiperazine” or “DKP” includes diketopiperazines and salts, derivatives, analogs and modifications thereof falling within the scope of the general Formula 1, wherein the ring atoms E₁ and E₂ at positions 1 and 4 are either O or N and at least one of the side-chains R₁ and R₂ located at positions 3 and 6 respectively contains a carboxylic acid (carboxylate) group. Compounds according to Formula 1 include, without limitation, diketopiperazines, diketomorpholines and diketodioxanes and their substitution analogs. For exemplary purposes the preferred embodiment, diketopiperazines and their derivatives, will be described in detail; however, it is understood that this is not to the exception of other heterocyclic compounds based on Formula 1.

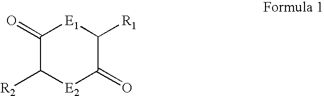

Diketopiperazines, in addition to making aerodynamically suitable microparticles, also facilitate transport across cell layers, further speeding absorption into the circulation. Diketopiperazines can be formed into particles that incorporate a drug or particles onto which a drug can be adsorbed. The combination of a drug and a diketopiperazine can impart improved drug stability. These particles can be administered by various routes of administration. As dry powders these particles can be delivered by inhalation to specific areas of the respiratory system, depending on particle size. Additionally, the particles can be made small enough for incorporation into an intravenous suspension dosage form. Oral delivery is also possible with the particles incorporated into a suspension, tablets or capsules. Diketopiperazines may also facilitate absorption of an associated drug.

In another embodiment of the present invention, the DKP is a derivative of 3,6-di(4-aminobutyl)-2,5-diketopiperazine, which can be formed by (thermal) condensation of the amino acid lysine. Exemplary derivatives include 3,6-di(succinyl-4-aminobutyl)-, 3,6-di(maleyl-4-aminobutyl)-, 3,6-di(glutaryl-4-aminobutyl)-, 3,6-di(malonyl-4-aminobutyl)-, 3,6-di(oxalyl-4-aminobutyl)-, and 3,6-di(fumaryl-4-aminobutyl)-2,5-diketopiperazine (hereinafter fumaryl diketopiperazine or FDKP). The use of DKPs for drug delivery is known in the art (see for example U.S. Pat. No. 5,352,461 entitled “Self Assembling Diketopiperazine Drug Delivery System”; U.S. Pat. No. 5,503,852 entitled “Method For Making Self-Assembling Diketopiperazine Drug Delivery System”; U.S. Pat. No. 6,071,497 entitled “Microparticles For Lung Delivery Comprising Diketopiperazine”; and U.S. Pat. No. 6,331,318 entitled “Carbon-Substituted Diketopiperazine Delivery System”, each of which is incorporated herein by reference in its entirety for all that it teaches regarding diketopiperazines and diketopiperazine-mediated drug delivery). The use of DKP salts is described in co-pending U.S. patent application Ser. No. ______ filed Aug. 23, 2005 and entitled “Diketopiperazine Salts For Drug Delivery And Related Methods” and known to all by U.S. Provisional Patent Application No. 60/603,761, which is hereby incorporated by reference in its entirety. Pulmonary drug delivery using DKP microparticles is disclosed in U.S. Pat. No. 6,428,771 entitled “Method For Drug Delivery To The Pulmonary System”, which is hereby incorporated by reference in its entirety.

As used herein, the term “microparticles” includes microcapsules having an outer shell composed of either a diketopiperazine alone or a combination of a diketopiperazine and one or more drugs. It also includes microspheres containing drug dispersed throughout the sphere; particles of irregular shape; and particles in which the drug is coated in the surface(s) of the particle or fills voids therein.

To combine PDE5 inhibitors with a DKP several alternatives are available. In one embodiment of the present invention, a DKP salt of a PDE5 inhibitor is produced. In non-limiting example, sildenafil is currently sold as a citrate salt. An anionic DKP, such as FDKP, can be substituted for the citrate to make the FDKP salt of sildenafil. The FDKP salt of sildenafil could be prepared by dissolving both sildenafil and FDKP in an appropriate solvent in the appropriate ratio. Solvent removal by, for example, evaporation, lyophilization, or spray drying would provide the isolated salt as an oil or dry powder. Similarly, salts incorporating other PDE5 inhibitors (e.g., tadalafil, vardenafil, and the like) or other substituted DKPs can also be made.

In another embodiment of the present invention, microparticles combining a DKP and a PDE5 inhibitor, or salt thereof, are prepared by spray drying a solution of the PDE5 inhibitor, or salt thereof, and DKP or by spray drying a solution of the PDE5 inhibitor, or salt thereof, in which DKP microparticles are suspended. Such solutions could also be lyophilized. Depending in part on the concentration of the solution, a suitable dry powder can be obtained directly (see for example U.S. Pat. No. 6,440,463 entitled “Methods For Fine Powder Formation”, which is hereby incorporated by reference in its entirety). Alternatively, the solid obtained can be micronized to obtain particles of a suitable size. For pulmonary administration, particles of less than about 10μ are desired, preferably less than about 5μ, and more preferably about 1 μto about 3μ.

In one embodiment, PDE5 inhibitors, or salts thereof, are associated with microparticles by dissolving a DKP with acidic R groups in bicarbonate or other basic solution, adding the active agent in solution or suspension, and then precipitating the microparticle by adding acid, such as 1 M citric acid.

In another embodiment, PDE5 inhibitors, or salts thereof, are associated with microparticles by dissolving a DKP with basic R groups in an acidic solution, such as 1 M citric acid, adding the active agent in solution or suspension, and then precipitating the microparticle by adding bicarbonate or another basic solution.

In still another embodiment, PDE5 inhibitors, or salts thereof, are associated with microparticles by dissolving a DKP with both acidic and basic R groups in an acidic or basic solution, adding the active agent in solution or suspension to be encapsulated, then precipitating the microparticle by neutralizing the solution.

The microparticles can be stored in the dried state and suspended for administration to a patient. In a first embodiment, the reconstituted microparticles maintain their stability in an acidic medium and dissociate as the medium approaches physiological pH in the range of between 6 and 14. In a second embodiment, suspended microparticles maintain their stability in a basic medium and dissociate at a pH of between 0 and 6. In a third embodiment, the reconstituted microparticles maintain their stability in an acidic or basic medium and dissociate as the medium approaches physiological pH in the range of pH between 6 and 8.

The impurities typically are removed when the microparticles are precipitated. However, impurities also can be removed by washing the particles to dissolve the impurities. A preferred wash solution is water or an aqueous buffer. Solvents other than water also can be used to wash the microspheres or precipitate the DKPs, in order to remove impurities that are not water soluble. Any solvent in which neither the PDE5 inhibitor, or salt thereof, nor the DKP is soluble are suitable. Examples include acetic acid, ethanol, and toluene.

In an alternative embodiment, microparticles of DKP are prepared and provided in a suspension, typically an aqueous suspension, to which a solution of the PDE5 inhibitors, or salts thereof, are then is added. The suspension is then lyophilized or freeze dried to yield DKP microparticles having a coating of PDE5 inhibitor.

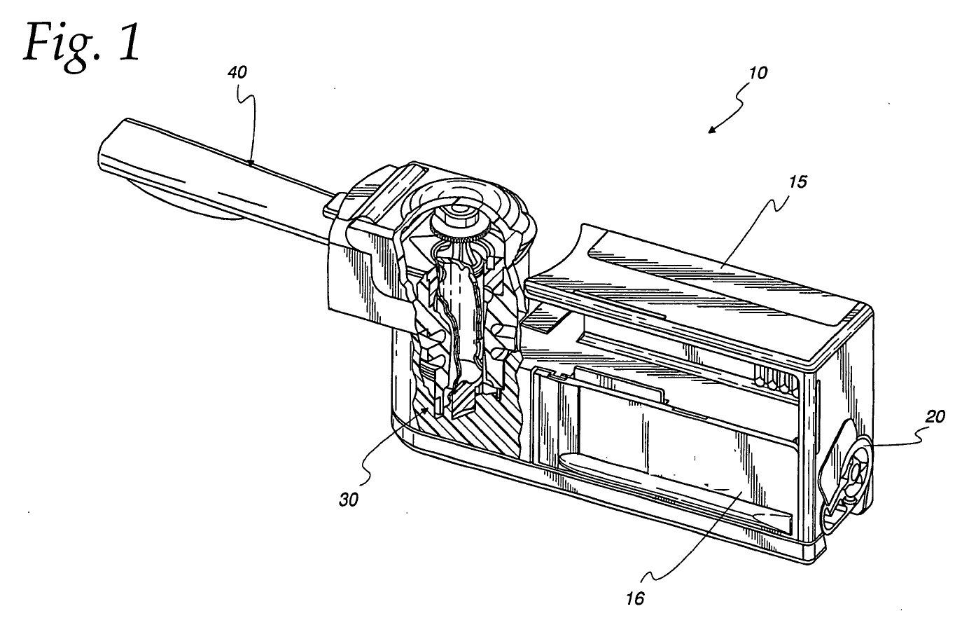

Pulmonary delivery can be very effectively accomplished using dry powders comprising the microparticles of the invention and can lead to rapid absorption into the circulation (bloodstream). Once a dry powder is obtained it can be administered using a variety of dry powder inhalers commercially available or otherwise known in the art. Particularly suitable inhaler systems are described in U.S. patent application Ser. Nos. 09/621,092 and 10/655,153, both entitled “Unit Dose Capsules And Dry Powder Inhaler”, which are hereby incorporated by reference in their entirety. The drug powder inhaler claimed in the above referenced pending patent applications is depicted in FIG. 1.

FIG. 1 shows an embodiment of a dry powder inhaler 10 suitable for delivering the compositions described herein to the pulmonary system. In broad conceptual terms, an inhaler housing 15 includes an intake section 20, a mixing section 30 and a mouthpiece 40. In the preferred embodiment, this inhaler housing 15 is approximately 93 mm long, 38 mm high, and 22 mm thick. The other parts illustrated and described here are of proportionate size. The mouthpiece 40 can be swiveled from a stored position within the housing 15 to a cartridge installation position in which the mouthpiece 40 is oriented at 90 degrees to the long dimension of the housing. When a cap 352 is closed, the mouthpiece can then be further rotated into an operating position in which the mouthpiece is located at a 180 degree position to the long dimension of the housing. When the mouthpiece 40 is stored within the inhaler 15, a sliding dirt shield cover 16 slidably mounted stored on the housing can be slid upwardly to protect the mouthpiece 40 and the air intake conduit entrance of the inhaler. The housing 15 can be formed of a gamma radiation-proof polycarbonate plastic for the rapid sterilization of the inhaler in mass production, as well as in clinical-hospital use. A cartridge containing a powder formulation of a composition of the present invention is inserted in mixing chamber 30 for pulmonary delivery of the composition.

Diketopiperazine salts of PDE5 inhibitors or microparticles having PDE5 inhibitors associated therewith are suitable for oral administration, for example, as tablets, pills, capsules, or troches. These microparticles, depending on the chemical nature and size, will either be absorbed to, or passed through, the epithelial lining of the gastrointestinal tract into the bloodstream or lymphatic system. These can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier. In addition, dosage unit forms can contain various other materials which modify the physical form of the dosage unit, for example, coatings of sugar, shellac, or other enteric agents.

In another embodiment of the present invention a method is provided for treating sexual dysfunction comprising delivering to the pulmonary system of a patient in need of treatment for sexual dysfunction, a DKP salt of a PDE5 inhibitor or DKP microparticles comprising a PDE5 inhibitor or a salt thereof.

To treat a patient for sexual dysfunction, the patient simply inhales the composition of the present invention before erectile function is desired at the time of a sexual encounter in a pharmacologically active amount sufficient to achieve vasodilation. Physicians and pharmacologists of ordinary skill in the art are knowledgeable in titrating doses to obtain the amount sufficient to achieve the desired clinical endpoint. A pharmacologically sufficient amount of drug is a dose that achieves the desirable clinical endpoint but does not have a undesirable side effects at a level which would result in the cessation of treatment. Typical doses for the pulmonary drug delivery of the present invention can be from about 0.1 to about 100 mg, depending on the particular drug being used. Preferably the dose delivered to the alveolar surface is in the range of from about 0.5 to about 50 mg. Although conventional oral PDE5 inhibitor formulations do not produce efficacious, systemic concentrations of the drug until several hours after administration, an oral formulation that provides a rapid onset of action is nonetheless desirable as an alternative to pulmonary delivery. A rapid-acting formulation can be prepared by use of an agent, such as a DKP, that facilitates rapid drug absorption following oral administration. Thus, an oral dosage form containing, for example, a combination of FDKP and sildenafil, either as a salt or a physical mixture, can provide a rapid onset of drug action.

Sexual dysfunction exists in many forms and can be classified into two classes, male sexual dysfunction and female sexual dysfunction. The most common form of male sexual dysfunction is erectile dysfunction. Female sexual dysfunction can be due to a variety of causes including, but not limited to, antidepressant-induced sexual dysfunction, sexual dysfunction secondary to multiple sclerosis, anorgasmia, low arousal, delayed orgasm, decreased vaginal engorgement, dyspareunia or infertility-induced sexual dysfunction.

In one embodiment of the present invention, a method is provided for treating pulmonary hypertension comprising delivering to the pulmonary system of a patient in need of treatment for pulmonary hypertension, a DKP salt of a PDE5 inhibitor or DKP microparticles comprising a PDE5 inhibitor or a salt thereof. For treatment of pulmonary hypertension the patient would take a dose of 0.5 to 50 mg one to six times daily. The ability to administer a therapeutically active drug directly to the internal surfaces of the lung is particularly important to the pathology of pulmonary hypertension. As compared to systemic administration, pulmonary administration can provide a significant improvement and efficiency in the treatment of this life threatening disorder.

Pulmonary hypertension is a rare blood vessel disorder of the lung in which the pressure in the pulmonary artery (the blood vessel that leads from the heart to the lungs) rises above normal levels and may become life threatening. Symptoms of pulmonary hypertension include shortness of breath with minimal exertion, fatigue, chest pain, dizzy spells and fainting. When pulmonary hypertension occurs in the absence of a known cause, it is referred to as primary pulmonary hypertension (PPH). This term should not be construed to mean that because it has a single name it is a single disease. There are likely many unknown causes of PPH. PPH is extremely rare, occurring in about two persons per million population per year.

Secondary pulmonary hypertension (SPH) means the cause is known. Common causes of SPH include the breathing disorders emphysema and bronchitis. Other less frequent causes are the inflammatory or collagen vascular diseases such as scleroderma, CREST syndrome or systemic lupus erythematosus (SLE). Congenital heart diseases that cause shunting of extra blood through the lungs like ventricular and atrial septal defects, chronic pulmonary thromboembolism (old blood clots in the pulmonary artery), HIV infection, liver disease and diet drugs like fenfluramine and dexfenfluramine are also causes of pulmonary hypertension.

Many forms of pulmonary hypertension are suitable for treatment with the compositions of the present invention including, but not limited to, primary pulmonary hypertension (PPH), acute pulmonary hypertension, pulmonary arterial hypertension (PAH), pregnancy-associated hypertension such as preeclampsia, and persistent pulmonary hypertension of the newborn (PPHN).

### EXAMPLES

### Example 1

### Preparation of the FDKP Salt of Sildenafil—Method 1

Thirteen grams of FDKP (28.73 mmol, 1 equiv.) are placed into a 250 mL 3-neck round bottom flask equipped with a reflux condenser, magnetic stir bar, and thermometer. The reaction is run under a nitrogen atmosphere. Water (150 mL) and sildenafil (13.6 g, 1 equiv.) are added sequentially to the flask. The resulting yellow solution is heated to 50° C. and held for 2 hours. The solution is hot filtered to remove any insoluble material. The water is removed from the sample via rotary evaporation. The recovered solids are dried in a vacuum oven (50° C., 30 inches of mercury) overnight. The salt is then assayed for moisture content (Karl Fischer) and sodium content (elemental analysis and titration). The yield of the salt is typically from about 90% to about 95%, by weight.

### Example 2

### Preparation of the FDKP Salt of Sildenafil—Method 2

Thirteen grams of FDKP (28.73 mmol, 1 equiv.) and ethanol (150 mL) are placed into a 250 mL 3-neck round bottom flask equipped with a reflux condenser, magnetic stir bar, and thermometer. The reaction is run under a nitrogen atmosphere. The slurry is heated to 50° C. Sildenafil (13.6 g, 1 equiv.) is added in one portion. The resulting slurry is held at 50° C. for 2 hours. The reaction contents are cooled to ambient temperature (20° C. to 30° C.) and the solids isolated by vacuum filtration. The recovered salt is washed with ethanol (300 mL) and acetone (150 mL) and dried in the vacuum oven (50° C., 30 inches of mercury) overnight. No further purification is required. The salt is then assayed for moisture content (Karl Fischer) and sodium content (elemental analysis and titration). The yield of the salt is typically from about 90% to about 95%, by weight.

### Example 3

### Preparation of FDKP Microparticles Associated with Sildenafil

Sildenafil is associated with 2,5-diketo-3,6-di(4-fumarylaminobutyl)piperazine (FDKP) in microparticles by adding 1.6 grams of sildenafil to 320 mL of a 0.5% solution of sodium lauryl sulfate in 0.1M sodium bicarbonate. To this suspension is added 4 grams of 2,5-diketo-3,6-di(4-fumarylaminobutyl)piperazine. The final suspension is placed under a probe sonicator and sonicated over a one minute period while 320 mL of 0.1M citric acid is added. The suspension is sonicated for an additional five minutes at room temperature, at which time precipitation of the microparticles is complete. The particles are isolated by centrifugation at 10,000 rpm for ten minutes, and the sample is lyophilized at room temperature overnight. The yield after drying is determined.

The size of the PFE5-containing FDKP microparticles is determined by scanning electron microscopy (SEM), visible light microscopy with image analysis, laser light scattering, laser diffraction and Coulter counter techniques.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A composition comprising a diketopiperazine salt of a phosphodiesterase type 5 (PDE5) inhibitor.

2. The composition of claim 1 wherein said PDE5 inhibitor is a substituted pyrimidinone.

3. The composition of claim 2 wherein said substituted pyrimidinone is selected from the group consisting of sildenafil, vardenafil, tadalafil and analogues thereof.

4. The composition of claim 1 wherein said PDE5 inhibitor is a pyrazolopyrimidinone.

5. The composition of claim 4 wherein said pyrazolopyrimidinone is selected from the group consisting of sildenafil, vardenafil, and analogues thereof.

6. The composition of claim 1 wherein said diketopiperazine has the general structure: 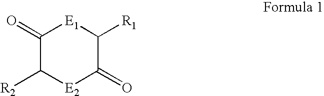
wherein ring atoms E₁ and E₂ are either O or N and at least one of R₁ and R₂ contain a carboxyl group.

7. The composition of claim 6 wherein both R₁ and R₂ contain a carboxyl group.

8. The composition of claim 6 wherein said diketopiperazine is selected from the group consisting of 2,5-diketo-3,6-di(4-fumarylaminobutyl)piperazine, 2,5-diketo-3,6-di(4-sucinylaminobutyl)piperazine, 2,5-diketo-3,6-di(4-glutarylaminobutyl)piperazine, and 2,5-diketo-3,6-di(4-maleylaminobutyl)piperazine.

9. The composition of claim 1 wherein the ratio of said PDE5 inhibitor to said diketopiperazine is about 1:1.

10. The composition of claim 1 wherein the ratio of said PDE5 inhibitor to said diketopiperazine is about 2:1.

11. The composition of claim 1 wherein said diketopiperazine salt is formulated as a dry microparticle.

12. A microparticle composition for delivery of a PDE5 inhibitor comprising:
diketopiperazine microparticles, wherein said microparticles are insoluble at a first defined pH and soluble at a second defined pH; and
a PDE5 inhibitor or a pharmaceutically acceptable salt thereof.

13. The microparticle composition of claim 12 wherein said PDE5 inhibitor or a pharmaceutically acceptable salt thereof is selected from the group consisting of sildenafil citrate, vardenafil hydrochloride and tadalafil.

14. The microparticle composition of claim 12 wherein said microparticle is formed by precipitation of a PDE5 inhibitor or a pharmaceutically acceptable salt thereof onto diketopiperazine microparticles.

15. The microparticle composition of claim 14 wherein said precipitation is initiated by freezing or chilling.

16. The microparticle composition of claim 12 wherein said microparticle is formed by spray drying diketopiperazine microparticles suspended in a solution of a PDE5 inhibitor or a pharmaceutically acceptable salt thereof.

17. The microparticle composition of claim 12 wherein said pharmaceutically acceptable salt is a diketopiperazine salt.

18. The microparticle composition of claim 12 wherein said microparticle is formed by precipitation of a solution comprising a diketopiperazine and a PDE5 inhibitor or a pharmaceutically acceptable salt thereof.

19. The microparticle composition of claim 12 wherein said diketopiperazine microparticles are formulated for delivery to the pulmonary system.

20. The microparticle composition of claim 12 wherein said diketopiperazine microparticles have a diameter between 0.5 microns and 10 microns and which release incorporated PDE5 inhibitor or a pharmaceutically acceptable salt thereof at a pH of 6.0 or greater.

21. The microparticle composition of claim 12 wherein said diketopiperazine microparticles are formulated for oral administration.

22. A method of treating sexual dysfunction comprising administering to a patient in need of treatment for sexual dysfunction, a composition comprising a DKP salt of a PDE5 inhibitor or DKP microparticles associated with a PDE5 inhibitor.

23. The method of claim 22 wherein the sexual dysfunction is erectile dysfunction.

24. The method of claim 22 wherein the sexual dysfunction is female sexual dysfunction.

25. The method of claim 24 wherein the sexual dysfunction is selected from the group consisting of antidepressant-induced sexual dysfunction, sexual dysfunction secondary to multiple sclerosis, anorgasmia, low arousal, delayed orgasm, decreased vaginal engorgement, dyspareunia and infertility-induced sexual dysfunction.

26. The method of claim 22 wherein said microparticles are delivered to the pulmonary system.

27. The method of claim 22 wherein said microparticles are administered orally.

28. A method of treating pulmonary hypertension comprising delivering to a patient in need of treatment for pulmonary hypertension, a composition comprising a DKP salt of a PDE5 inhibitor or DKP microparticles associated with a PDE5 inhibitor.

29. The method of claim 28 wherein said pulmonary hypertension is selected from the group consisting of primary pulmonary hypertension, acute pulmonary hypertension, pulmonary arterial hypertension, pregnancy-associated hypertension such as preeclampsia, and persistent pulmonary hypertension of the newborn.

30. The method of claim 28 wherein said microparticles are delivered to the pulmonary system.

31. The method of claim 28 wherein said microparticles are administered orally.
